(19) United States
(12) Patent Application Publication
(10) Pub. No.: US 2007/0231409 A1
(43) Pub. Date: Oct. 4, 2007

(54) Drug and/or food supplement containing NISYLEN®, CEPA, Euphrasia, Belladonna and/or Mercurius solubilis

(76) Inventor: Klaus Huber, Bad Tolz (DE)

Correspondence Address: ALLEMAN HALL MCCOY RUSSELL & TUTTLE LLP, 806 SW BROADWAY, SUITE 600, PORTLAND, OR, 97205-3335, US

(21) Appl. No.: 11/496,756

(22) Filed: Jul. 31, 2006

(30) Foreign Application Priority Data
Mar. 28, 2006 (DE) 20 2006 004 962.2
Mar. 30, 2006 (EP) 06 006 727.9

(51) Int. Cl. A61K 36/47; A61K 33/28

(52) U.S. Cl. 424/644; 424/725

(57) ABSTRACT

The present disclosure relates to a drug and/or food supplement with antiviral effects, in particular for the preventive and/or therapeutic treatment of herpes labialis and/or herpes genitalis, and/or with antibacterial and/or immunomodulating and/or antiphlogistic effects and/or for the preventive and/or therapeutic treatment of affections of the prostate, containing NISYLEN® and/or Cepa and/or Euphrasia and/or Belladonna and/or Mercurius solubilis.

### CROSS-REFERENCE TO RELATED APPLICATIONS

This application claims priority to German Patent Application Serial No. 20 2006 004 962.2 filed Mar. 28, 2006, and also claims priority to European Patent Application Serial No. 06 006 727.9 filed Mar. 30, 2006, both of which are hereby incorporated by reference in their entirety for all purposes.

### FIELD

The present disclosure relates to a drug and/or food supplement containing NISYLEN®, Cepa, Euphrasia., Belladonna and/or Mercurius solubilis and to the use of NISYLEN® and/or Cepa and/or Euphrasia and/or Belladonna and/or Mercurius solubilis for a drug and/or food supplement and for the manufacture thereof.

### BACKGROUND AND SUMMARY

NISYLEN® is known as a drug for use against influenza infections, fever, sore throat, headache, and cough. It is a homeopathic drug in the form of a compound preparation comprising various active substances, which is available in the form of tablets or a solution for oral administration. The active substances include Aconitum Dil. D3, Gelsemium Dil. D3, Ipecacuanha Dil. D3, Phosphorus Dil. D5, Bryonia Dil. D2 and Eupatorium perfoliatum Dil. D1. Aconitum is an extract of Actonitum, which is a flowering plant belonging to the buttercup family. Gelsemium is an extract of Gelsemium, which is a flowering plant belonging to the family of Gelsemiaceae. Ipecacuanha is an extract of Ipecacuanha, which is a flowering plant in the family of Rubiaceae, the root of which is most commonly used to make syrup of ipecac, a powerful emetic. Phosphorus is a homeopathic remedy derived from phosphorus, such as. potassium phosphate or magnesium phosphate. Bryonia is an extract from Bryony, which is a perennial, tendril-climbing flowering plant in the genus of Bryonia. Eupatorium perfoliatum is an extract of Eupatorium perfoliatum, which is a poisonous herbaceous perennial flowering plant.

Homeopathic active ingredients are often diluted with diluents, such as water and alcohol. Dil. 1D indicates that the active ingredient is diluted to 1/10 of its original strength. Dil 2D indicates that the active ingredient is diluted to 1/100 of its original strength. Dil 3D indicates that the active ingredient is diluted to 1/1000 of its original strength, and so forth.

What is likewise known is the use of Cepa, Euphrasia, Belladonna and Mercurius solubilis for homeopathic drugs.

It is the object underlying the present disclosure to indicate further medical uses of one or more of the ingredients stated above.

The present disclosure is based on the knowledge that NISYLEN® and/or Cepa and/or Euphrasia and/or Belladonna and/or Mercurius solubilis have antiviral effects, in particular in the preventive and/or therapeutic treatment of herpes labialis and/or herpes genitalis, and/or antibacterial and/or immunomodulating and/or antiphlogistic, i.e. anti-inflammatory effects and/or can be used for the preventive and/or therapeutic treatment of affections of the prostate, in particular for the treatment of prostatitis or of degenerations of the prostate.

Herpes labialis is a viral disease which is caused by the type I herpes simplex virus (HSV Type I), where small, oozing vesicles are formed on the lips, which are sore, cause pain and itching, and are highly infectious. It may also be referred to as a cold sore.

Herpes genitalis is caused by the type II herpes simplex virus (HSV Type II). In the case of herpes genitalis there are also formed vesicles filled with liquid, which cause considerable pain, neuralgias and itching, and are likewise highly infectious.

Both types of virus (HSV Types I and II) affect the skin, mucous membranes, eyes, the nervous system and in rare cases also inner organs. Due to the fact that the virus is constantly present in the body, relapses, i.e. new outbreaks of the disease, can occur again and again. The medicaments known at present are virostatics (which inhibit the growth of the virus but do not necessarily kill the virus), which shorten the course of the disease and alleviate the symptoms.

It has now been found in accordance with the present disclosure that a drug or food supplement which contains NISYLEN® and/or Cepa and/or Euphrasia and/or Belladonna and/or Mercurius solubilis exhibits antiviral effects and can be used for instance for the preventive and therapeutic treatment of herpes. Furthermore, it was found in accordance with the present disclosure that a drug or food supplement which contains NISYLEN® and/or Cepa and/or Euphrasia and/or Belladonna and/or Mercurius solubilis exhibits antibacterial and/or immunomodulating and/or antiphlogistic effects. Furthermore, it was found in accordance with the present disclosure that a drug or food supplement which contains NISYLEN® and/or Cepa and/or Euphrasia and/or Belladonna and/or Mercurius solubilis can be used for the preventive and/or therapeutic treatment of affections of the prostate, such as prostatitis or degenerations of the prostate.

The explanations given below concerning the inventive drug correspondingly apply to the food supplement in accordance with the present disclosure.

The treatment of patients suffering from herpes labialis and of patients suffering from herpes genitalis with the drug in accordance with the present disclosure has shown that the course of the disease has been shortened considerably in both cases. The treatment of patients suffering of affections of the prostate with the inventive drug has shown that not only the PSA value (PSA=prostate-specific antigen), but also the PSA quotient has been improved considerably. The quotient of free PSA and total PSA value as measured in the serum of patients at the time of diagnosis is regarded as a measure for the malignancy of a tumor, the smaller the quotient, the more malignant the tumor.

In accordance with the present disclosure it can be provided that the drug contains one of the active substances NISYLEN® or Cepa or Euphrasia or Belladonna or Mercurius solubilis as main ingredient and one or more of the further ingredients as secondary ingredients, the main ingredient being present in a larger amount or concentration than any of the further ingredients.

It is conceivable in principle that the drug contains only NISYLEN®, only Cepa, only Euphrasia, only Belladonna or only Mercurius solubilis as the active substance.

The present disclosure comprises any combinations of the ingredients mentioned above, for instance a drug comprising NISYLEN® as main ingredient and all the others of said ingredients of the combinations thereof as secondary ingredients.

Preferably, it is provided that the drug contains NISYLEN®.

In accordance with a another aspect it is provided that the drug furthermore contains Cepa. Cepa is an extract from the plant Cepa (or Allium Cepa). Cepa likewise is a homeopathically active substance, which generally is used against a cold.

In accordance with a further aspect of the present disclosure, the drug furthermore contains Euphrasia. Euphrasia is an extract from the plant Euphrasia, which is herbaceous flowing plant in the family of Orobanchaceae. Euphrasia likewise is a substance known from homeopathy, which generally is used against affections of the eyes.

Additional ingredients of the drug in accordance with the present disclosure can be Belladonna and/or Mercurius solubilis. Belladonna is an extract from the plant belladonna, which is a member of the nightshade family. At present, Belladonna is used as a homeopathic medicament against various forms of inflammations. Mercurius solubilis is a homeopathic medicament which is known for the treatment of, for instance, sore throat, otitis media and hoarseness. Mercurius solubilis is the nitrate salt of mercury and can be prepared by dissolving liquid mercury in nitric acid.

In accordance with a further aspect of the present disclosure it is possible that—as far as contained in the drug—the ingredients Cepa, Euphrasia and Mercurius solubilis are present in the dilution D12 and Belladonna in the dilution D15. In principle, it is likewise possible to use the active substances in other dilutions.

It can be particularly advantageous when the ingredient NISYLEN® is present in the drug in a larger amount or concentration than the further ingredients.

Preferably, the drug contains NISYLEN®, Cepa, Euphrasia, Belladonna and Mercurius solubilis, NISYLEN® being present in the drug in a larger amount than the other ingredients mentioned above.

Particularly advantageously, the ingredient NISYLEN® is present in two to four times, preferably three times the amount of any of the further active substances of the drug.

In accordance with a further aspect of the invention it is provided that the drug contains NISYLEN® in a weight or in a volume of 4.0 to 4.5 g or ml, preferably in a weight or volume of 4.286 g or ml, and Cepa, Euphrasia, Belladonna and Mercurius solubilis each in a weight or in a volume of 1.2 to 1.6 g or ml, preferably in a weight or in a volume of 1.429 g or ml per 10 ml or 10 g of solution or drug or food supplement.

It is conceivable to prepare the drug for oral administration and/or for topical application. The latter can for instance be relevant in the treatment of herpes labialis.

In accordance with a preferred aspect, the drug is present in liquid form, which can be an alcoholic solution for oral administration. Other solutions than alcohol are conceivable as well. In principle it is likewise conceivable to provide the drug in solid form, for instance as powder, tablets or globuli. It is also possible to provide the drug in pasty form or as an ointment.

The drug preferably is a homeopathic drug.

The ingredient NISYLEN® can contain the following ingredients: Aconitum napellus, Gelsemium sempervirens, Cephaelis ipecacuanha, Phosphorus, Bryonia and Eupatorium perfoliatum. It is conceivable that per 1 g of NISYLEN® the ingredients are included in the following amounts: 100 mg of Aconitum napellus D3, 100 mg of Gelsemium sempervirens D3, 100 mg of Cephaelis ipecacuanha D3, 100 mg of Phosphorus D5, 100 mg of Bryonia D2, and 100 mg of Eupatorium perfoliatum D1.

Further details and advantages of the invention will be explained in detail with reference to an embodiment illustrated below. Also, the drug and/or supplement can be packaged with instructions indicating the potential uses or ailments for which it may be taken, such as one or more of those noted herein.

The drug in accordance with the embodiment described here contains the following ingredients:

| | | |
|---|---|---|
| NISYLEN ®: | 4.286 | g |
| Cepa D12: | 1.429 | g |
| Euphrasia D12: | 1.429 | g |
| Belladonna D15: | 1.429 | g |
| Mercurius solubilis D12: | 1.429 | g |

These ingredients are present in an alcoholic solution in a volume of 10 ml.

As stated above, such a solution is, among other things, excellently suited for the treatment of herpes genitalis and of herpes labialis as well as for the treatment of affections of the prostate.

It can be provided to take for instance 3×5 drops per day in the case of adults and 3×2 drops per day in the case of children. Particularly preferably, the drug should be taken regularly.

In the acute stage, or preferably in the prodromal stage, a half-hourly to hourly application is conceivable as well, i.e. per half an hour to an hour e.g. 5 drops (adults) or 2 drops (children) of the solution should be taken.

It can be advantageous not to dilute the solution and keep it in the mouth as long as possible. It furthermore turns out to be advantageous to drink much, if possible about 3 l/day.

As stated above, the above-mentioned herpes diseases frequently have relapses, which can occur once or several times a year, possibly also every month. The medicaments known so far are helpful in the acute stage by shortening the course of the disease and possibly by alleviating the symptoms, but cannot prevent the occurrence of relapses. In addition, resistences to the known medicaments have meanwhile occurred.

The present disclosure opens up the possibility to achieve an effective treatment of herpes genitalis as well as herpes labialis by means of a particularly effective medicament/food supplement. An effective treatment of affections of the prostate, in particular of prostatitis or degenerations of the prostate, is possible as well.

In principle, deviations from the embodiment of the present disclosure described above in detail are comprised as well. The solvent need not be an alcoholic solvent. In principle, other suitable solvents can be used as well. As stated above, the present disclosure furthermore is not restricted to the application as liquid medicament. It is likewise conceivable to provide the medicament in solid form (tablets, powder, globuli) or as an ointment.

1. A drug and/or food supplement with antiviral effects comprising NISYLEN® and/or Cepa and/or Euphrasia and/or Belladonna and/or Mercurius solubilis.

2. The drug and/or food supplement as claimed in claim 1, wherein the drug or food supplement performs the preventive and/or therapeutic treatment of herpes labialis and/or herpes genitalis, and/or with antibacterial and/or immunomodulating and/or antiphlogistic effects and/or performs preventive and/or therapeutic treatment of affections of the prostate.

3. The drug and/or food supplement as claimed in claim 2, wherein it includes NISYLEN® as a main ingredient and one or more of the further ingredients as secondary ingredients.

4. The drug and/or food supplement as claimed in claim 2, wherein the drug includes Cepa as a main ingredient and one or more of the further ingredients as secondary ingredients.

5. The drug and/or food supplement as claimed in claim 2, wherein it contains Euphrasia as a main ingredient and one or more of the further ingredients as secondary ingredients.

6. The drug and/or food supplement as claimed in claim 2, wherein it contains Belladonna as a main ingredient and one or more of the further ingredients as secondary ingredients.

7. The drug and/or food supplement as claimed in claim 2, wherein it contains Mercurius solubilis as a main ingredient and one or more of the further ingredients as secondary ingredients.

8. The drug and/or food supplement as claimed in claim 2, further comprising instructions identifying use for treatment of the prostate is a prostatitis or degenerations of the prostate.

9. The drug and/or food supplement as claimed in claim 2, wherein the ingredients Cepa, Euphrasia and Mercurius solubilis are present in the dilution D12 and Belladonna in the dilution D15.

10. The drug and/or food supplement as claimed in claim 2, wherein it includes NISYLEN®, Cepa, Euphrasia, Belladonna and Mercurius solubilis.

11. The drug and/or food supplement as claimed in claim 10, wherein the ingredient NISYLEN® is present in two to four times the amount of any of the other ingredients.

12. The drug and/or food supplement as claimed in claim 10, wherein it includes NISYLEN® in a weight or in a volume of 4.0 to 4.5 g or ml, and Cepa, Euphrasia, Belladonna and Mercurius solubilis each in a weight or in a volume of 1.2 to 1.6 g or ml per 10 ml of solution.

13. The drug and/or food supplement as claimed in claim 10, wherein it is present in liquid form.

14. The drug and/or food supplement as claimed in claim 13, wherein it is an alcoholic solution.

15. The drug and/or food supplement as claimed in claim 2, wherein it is present in solid or pasty form.

16. The drug and/or food supplement as claimed in claim 2, wherein the ingredient NISYLEN® includes the following ingredients: Aconitum napellus, Gelsemium sempervirens, Cephaelis ipecacuanha, Phosphorus, Bryonia and Eupatorium perfoliatum.

17. The drug and/or food supplement as claimed in claim 16, wherein per 1 g of NISYLEN® the ingredients are included in the following amounts: 100 mg of Aconitum napellus D3, 100 mg of Gelsemium sempervirens D3, 100 mg Cephaelis ipecacuanha D3, 100 mg of Phosphorus D5, 100 mg of Bryonia D2 and 100 mg of Eupatorium perfoliatum D1.

18. A method, comprising:
manufacturing a drug and/or food supplement with antiviral effects, said manufacturing including using NISYLEN® and/or Cepa and/or Euphrasia and/or Belladonna and/or Mercurius solubilis.

19. The method of claim 18, wherein said drug and/or food supplement preventively and/or therapeutically treats herpes labialis and/or herpes genitalis, and/or said drug and/or food supplement includes antibacterial and/or immunomodulating and/or antiphlogistic effects, and/or preventively and/or therapeutically treats affections of the prostate

20. A method, comprising
preventing and/or therapeutically treating herpes labialis and/or herpes genitalis by using NISYLEN® and/or Cepa and/or Euphrasia and/or Belladonna and/or Mercurius solubilis as a drug and/or food supplement with antiviral effects.

21. A method, comprising
preventing and/or therapeutically treating affections of the prostate by using NISYLEN® and/or Cepa and/or Euphrasia and/or Belladonna and/or Mercurius solubilis as a drug and/or food supplement and/or with antibacterial and/or immunomodulating and/or antiphlogistic effects.

22. A method, comprising
preventing and/or therapeutically treating a virus in a human by using a drug and/or food supplement having antiviral effects, the drug and/or food supplement comprising NISYLEN® and/or Cepa and/or Euphrasia and/or Belladonna and/or Mercurius solubilis.

23. The drug and/or food supplement as claimed in claim 10, where NISYLEN® is present in a larger amount than Cepa, Euphrasia, Belladonna and Mercurius solubilis.
